(19) United States
(12) Patent Application Publication
(10) Pub. No.: US 2021/0220459 A1
(43) Pub. Date: Jul. 22, 2021

(54) VACCINE COMPOSITIONS

(71) Applicants: The University of Sydney, New South Wales (AU); Meat & Livestock Australia Limited, North Sydney, New South Wales (AU)

(72) Inventors: Richard WHITTINGTON, New South Wales (AU); Auriol PURDIE, New South Wales (AU); Kumudika DE SILVA, New South Wales (AU); Karren PLAIN, New South Wales (AU); Douglas BEGG, New South Wales (AU); Om DHUNGYEL, New South Wales (AU)

(73) Assignees: The University of Sydney, New South Wales (AU); Meat & Livestock Australia Limited, North Sydney, New South Wales (AU)

(21) Appl. No.: 16/760,842

(22) PCT Filed: Nov. 5, 2018
(86) PCT No.: PCT/AU2018/051195

§ 371 Date: Apr. 30, 2020

(30) Foreign Application Priority Data
Nov. 3, 2017 (GB) 1718251.0

(51) Int. Cl. A61K 39/04; A61K 39/39; A61K 9/107; A61K 47/22; A61P 31/04

(52) U.S. Cl. CPC A61K 39/04; A61K 39/39; A61K 2039/55566; A61K 47/22; A61P 31/04; A61K 9/107

(57) ABSTRACT

The present invention is directed to novel vaccine compositions and methods for immunising subjects against Mycobacterium avium subspecies paratuberculosis. The invention involves the use of mineral oil adjuvants, or white mineral oil adjuvants, more specifically those having CAS 8042-47-5, CAS 1335203-18-3, CAS 1174522-45-2, CAS 1335203-17-2 (or EC equivalents 232-455-8, 932-078-5, 934-954-2 and 934-956-3, respectively) to reduce lesions or adverse reactions.

### FIELD OF THE INVENTION

The present invention relates to novel compositions for use in vaccinating animals to minimise or reduce the severity of infection with Mycobacterium avium subspecies paratuberculosis.

### RELATED APPLICATION

This application claims priority from GB patent application no. 1718251.0, the entire contents of which are hereby incorporated by reference.

### BACKGROUND OF THE INVENTION

Johne's disease (also called paratuberculosis or JD), is a chronic, contagious infection with the bacterium Mycobacterium avium subspecies paratuberculosis (Mptb or MAP). The disease primarily affects ruminants, and is characterised by intermittent diarrhoea or softening of faeces, emaciation and eventually death.

Johne's disease can result in a significant economic impact for farmers. Currently, there is no treatment for JD, and vaccination is typically used as a key control measure. Vaccination to minimise the impact of MAP infection in cattle has been in use since the 1920s, with varying success.

While currently available commercial vaccines do not prevent infection with MAP, they are effective in reducing clinical signs of disease. Animals with active signs of infection also shed the organisms in their faeces, transmitting the infection to other animals. Vaccination can be helpful in reducing the level of faecal shedding and consequently, transmission among members of the ruminant population. In particular, reduction of faecal shedding can aid in reducing transmission to calves, who are more susceptible to acquiring infection (for example, as a result of swallowing small amounts of manure either from the udder of the mother or from the birthing environment). Newborns may also acquire infection while in the uterus or by swallowing bacteria passed in the milk or colostrum.

One disadvantage of existing vaccines available for immunising cattle against MAP infection is that the vaccines cause lesions to develop at the site of immunisation in a large proportion of animals. For example, it is thought that the combination of the immunogen in conjunction with the adjuvant induces a severe hypersensitivity reaction at the injection site, which can cause the formation of an abscess, then a persistent nodule (granuloma) and this can also occur in the regional lymph nodes. Occasional rupture of the nodule results in a fistula that causes suffering to the animal and potential downgrading of the carcass at slaughter, with concomitant diminished returns for the farmer. Another concern to users of the currently available vaccines is human safety, since recovery from accidental self-injection may take months, and may require multiple medical treatments, including surgery.

As such, there is a need to develop vaccines and immune stimulating compositions which can elicit an immune response to MAP in animals at risk of infection, but which are safer both for the animals, and the user.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention provides a vaccine or immune stimulating composition comprising:
an immunogen for providing an immune response to Mycobacterium avium subspecies paratuberculosis in a subject;
an adjuvant for potentiating the immune response to the immunogen in the subject;
thereby forming a vaccine or immune stimulating composition.

In preferred embodiments of the invention, the adjuvant for potentiating the immune response to the immunogen comprises, consists of or consists essentially of, a mineral oil, including a refined mineral oil as identified by the Chemical Abstract Service (CAS) no: 8042-47-5. The mineral oil CAS 8042-47-5 may also be referred to by the European number for chemicals (EC) no: 232-455-8.

Other mineral oils that are also suitable for use in the vaccine compositions of the present invention include those having the identifiers EC no: 932-078-5, EC no: 934-954-2 and EC no: 934-956-3.

In a particularly preferred embodiment, the adjuvant comprises the mineral oil identified by CAS no: 8042-47-5. As such, the invention also provides a vaccine or immune stimulating composition comprising:
an immunogen for providing an immune response to Mycobacterium avium subspecies paratuberculosis in a subject,
an adjuvant for potentiating the immune response to the immunogen, wherein the adjuvant comprises, consists or consists essentially of the mineral oil identified by CAS no: 8042-47-5
thereby forming a vaccine or immune stimulating composition.

The present invention also provides a vaccine or immune stimulating composition comprising:
an immunogen for providing an immune response to Mycobacterium avium subspecies paratuberculosis in a subject, wherein the immunogen is in the form of a plurality of killed Mycobacterium avium subspecies paratuberculosis organisms, and
an adjuvant for potentiating the immune response to the immunogen;

thereby forming a vaccine or immune stimulating composition.

The adjuvant may comprise, consist or consist essentially of the mineral oil having CAS no: 8042-47-5, or European number for chemicals (EC) no: 232-455-8, EC no: 932-078-5, EC no: 934-954-2 or EC no: 934-956-3.

The present invention thus provides a vaccine or immune stimulating composition comprising:
an immunogen for providing an immune response to Mycobacterium avium subspecies paratuberculosis in a subject, wherein the immunogen is in the form of a plurality of killed Mycobacterium avium subspecies paratuberculosis organisms, and
an adjuvant for potentiating the immune response to the immunogen, wherein the adjuvant comprises, consists or consists essentially of a mineral oil identified by CAS no: 8042-47-5

thereby forming a vaccine or immune stimulating composition.

Preferably, the present invention provides a vaccine or immune stimulating composition comprising:
an immunogen for providing an immune response to Mycobacterium avium subspecies paratuberculosis in a subject, wherein the immunogen is in the form of a plurality of killed Mycobacterium avium subspecies paratuberculosis organisms, and
an adjuvant for potentiating the immune response to the immunogen, wherein the adjuvant consists of a mineral oil identified by CAS no: 8042-47-5
thereby forming a vaccine or immune stimulating composition.

The plurality of killed Mycobacterium avium subspecies paratuberculosis organisms may include only one strain of Mycobacterium avium subspecies paratuberculosis, or the plurality may include a mixture of two or more different strains of Mycobacterium avium subspecies paratuberculosis.

Alternatively, the immunogen for providing the immune response may be selected from the group consisting of: a plurality of live attenuated Mycobacterium avium subspecies paratuberculosis organisms representative of one or more strains of the organism; a cell lysate formed from a plurality of Mycobacterium avium subspecies paratuberculosis organisms, including organisms of different strains; and one or more peptides or polypeptides having the sequence of a Mycobacterium avium subspecies paratuberculosis protein.

In still further embodiments, the vaccine or immune stimulating composition includes an emulsifier for emulsifying the immunogen and adjuvant such that the invention provides a vaccine or immune stimulating composition comprising:
an immunogen for providing an immune response to Mycobacterium avium subspecies paratuberculosis in a subject; the immunogen being provided in an aqueous medium;
an adjuvant for potentiating the immune response to the immunogen, wherein the adjuvant comprises a mineral oil;
an emulsifier for emulsifying the immunogen and adjuvant;

thereby forming a vaccine or immune stimulating composition.

In certain embodiments, the emulsifier is a mannide monooleate.

Accordingly, the invention provides a vaccine or immune stimulating composition comprising, consisting or consisting essentially of:
an immunogen for providing an immune response to Mycobacterium avium subspecies paratuberculosis in a subject, the immunogen being provided in an aqueous medium and wherein the immunogen is in the form of a plurality of killed Mycobacterium avium subspecies paratuberculosis organisms;
an adjuvant for potentiating the immune response to the immunogen, wherein the adjuvant comprises or consists of a mineral oil identified by CAS no: 8042-47-5; and
an emulsifier for emulsifying the immunogen and adjuvant;

thereby forming a vaccine or immune stimulating composition.

Further, the invention provides a vaccine or immune stimulating composition comprising, consisting or consisting essentially of:
an immunogen for providing an immune response to Mycobacterium avium subspecies paratuberculosis in a subject; the immunogen being provided in an aqueous medium;
an adjuvant for potentiating the immune response to the immunogen, wherein the adjuvant comprises or consists of a mineral oil identified by CAS no: 8042-47-5; and
an emulsifier in the form of a mannide monooleate for emulsifying the immunogen and adjuvant;

thereby forming a vaccine or immune stimulating composition.

Preferably, the invention provides a vaccine or immune stimulating composition comprising, consisting or consisting essentially of:
an immunogen for providing an immune response to Mycobacterium avium subspecies paratuberculosis in a subject; the immunogen being provided in an aqueous medium and wherein the immunogen is in the form of a plurality of killed Mycobacterium avium subspecies paratuberculosis organisms,
an adjuvant for potentiating the immune response to the immunogen, wherein the adjuvant comprises or consists of a mineral oil identified by CAS no: 8042-47-5; and
an emulsifier in the form of a mannide monooleate for emulsifying the immunogen and adjuvant;

thereby forming a vaccine or immune stimulating composition.

The present invention also provides a method for inducing an immune response to Mycobacterium avium subspecies paratuberculosis in a subject, the method comprising administering to a subject in need thereof, a vaccine or immune stimulating composition comprising:
an immunogen for providing an immune response to Mycobacterium avium subspecies paratuberculosis in a subject,
an adjuvant for potentiating the immune response to the immunogen in the subject;
   thereby inducing an immune response to Mycobacterium avium subspecies paratuberculosis in the subject.

The adjuvant may comprise the mineral oil having CAS no: 8042-47-5, or European number for chemicals (EC) no: 232-455-8, EC no: 932-078-5, EC no: 934-954-2 or EC no: 934-956-3. Preferably, the adjuvant for use in the method comprises the mineral oil having CAS no: 8042-47-5.

The present invention also provides a method of reducing the severity of infection with Mycobacterium avium subspecies paratuberculosis in a subject in need thereof, the method comprising administering to the subject, a vaccine or immune stimulating composition comprising:
an immunogen for providing an immune response to Mycobacterium avium subspecies paratuberculosis in a subject;
an adjuvant for potentiating the immune response to the immunogen;
   thereby reducing the severity of infection with Mycobacterium avium subspecies paratuberculosis in the subject.

The adjuvant preferably comprises or consists of the mineral oil having CAS no: 8042-47-5, or European number for chemicals (EC) no: 232-455-8, EC no: 932-078-5, EC no: 934-954-2 or EC no: 934-956-3. Preferably, the adjuvant for use in the method comprises the mineral oil having CAS no: 8042-47-5.

The present invention also provides a method of reducing the severity of one or more signs of Johne's Disease in a subject, the method comprising administering to the subject, a vaccine or immune stimulating composition comprising:
an immunogen for providing an immune response to Mycobacterium avium subspecies paratuberculosis in a subject,
an adjuvant for potentiating the immune response of the subject to the immunogen;
   thereby reducing the severity of one or more signs of Johne's Disease in the subject.

The adjuvant preferably comprises or consists of the mineral oil having CAS no: 8042-47-5, or European number for chemicals (EC) no: 232-455-8, EC no: 932-078-5, EC no: 934-954-2 or EC no: 934-956-3. Preferably, the adjuvant for use in the method comprises the mineral oil having CAS no: 8042-47-5.

Still further, the present invention provides a method of reducing the transmission of Mycobacterium avium subspecies paratuberculosis within a population of ruminants, the method comprising administering to one or more individuals in a population of ruminants, a vaccine or immune stimulating composition comprising:
an immunogen for providing an immune response to Mycobacterium avium subspecies paratuberculosis in the individual,
an adjuvant for potentiating the immune response to the immunogen, in the individual;
   thereby reducing the transmission of Mycobacterium avium subspecies paratuberculosis in the population.

The adjuvant preferably comprises or consists of the mineral oil having CAS no: 8042-47-5, or European number for chemicals (EC) no: 232-455-8, EC no: 932-078-5, EC no: 934-954-2 or EC no: 934-956-3. Preferably, the adjuvant for use in the method comprises the mineral oil having CAS no: 8042-47-5.

The present invention provides a method of reducing the transmission of Mycobacterium avium subspecies paratuberculosis within a population of ruminants, the method comprising administering to one or more individuals in a population of ruminants, a vaccine or immune stimulating composition comprising:
an immunogen for providing an immune response to Mycobacterium avium subspecies paratuberculosis in the individual,
an adjuvant for potentiating the immune response to the immunogen, in the individual, wherein the adjuvant comprises a mineral oil;
   thereby reducing the transmission of Mycobacterium avium subspecies paratuberculosis in the population.

The adjuvant preferably comprises or consists of the mineral oil having CAS no: 8042-47-5, or European number for chemicals (EC) no: 232-455-8, EC no: 932-078-5, EC no: 934-954-2 or EC no: 934-956-3. Preferably, the adjuvant for use in the method comprises the mineral oil having CAS no: 8042-47-5.

The present invention provides a method of reducing the transmission of Mycobacterium avium subspecies paratuberculosis within a population of ruminants, the method comprising administering to one or more individuals in a population of ruminants, a vaccine or immune stimulating composition comprising:
an immunogen for providing an immune response to Mycobacterium avium subspecies paratuberculosis in the individual,
an adjuvant for potentiating the immune response to the immunogen, in the individual, wherein the adjuvant comprises a mineral oil as identified by CAS no: 8042-47-5;
   thereby reducing the transmission of Mycobacterium avium subspecies paratuberculosis in the population.

The present invention also provides a kit for use in a method of:
reducing the severity of one or more signs of Johne's Disease in a subject;
inducing an immune response to Mycobacterium avium subspecies paratuberculosis in a subject;
reducing the transmission of Mycobacterium avium subspecies paratuberculosis within a population of ruminants;

the kit comprising
an immunogen for providing an immune response to Mycobacterium avium subspecies paratuberculosis in the individual,
an adjuvant for potentiating the immune response to the immunogen, in the individual, wherein the adjuvant comprises, consists or consists essentially of a mineral oil as identified by CAS no: 8042-47-5.

Optionally the kit comprises instructions for the use of the components.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Percentage of injection site lesions observed in the sheep in each treatment group across all time points for the different formulations of adjuvants with heat killed Mptb. Comparison of a single dose (the conventional method) or double dose (booster given 1 month after the primary vaccination and intended to provoke an adverse reaction) of the novel vaccine formulations to the positive control, Commercial Vaccine or unvaccinated animals. ‘A1’ refers to adjuvant 1 tested, ‘A2’ is adjuvant 2 etc. The adjuvant and antigen components are listed in Table 1. ‘NV’=no vaccine.

FIG. 2: Mptb-specific antibody responses from animals in the trial in which cattle were vaccinated with a prototype vaccine and inoculated. Error bars show the standard error of the mean.

FIG. 3: Mptb-specific IFNγ responses from the trial in which cattle were vaccinated with a novel vaccine and inoculated. Error bars show the standard error of the mean.

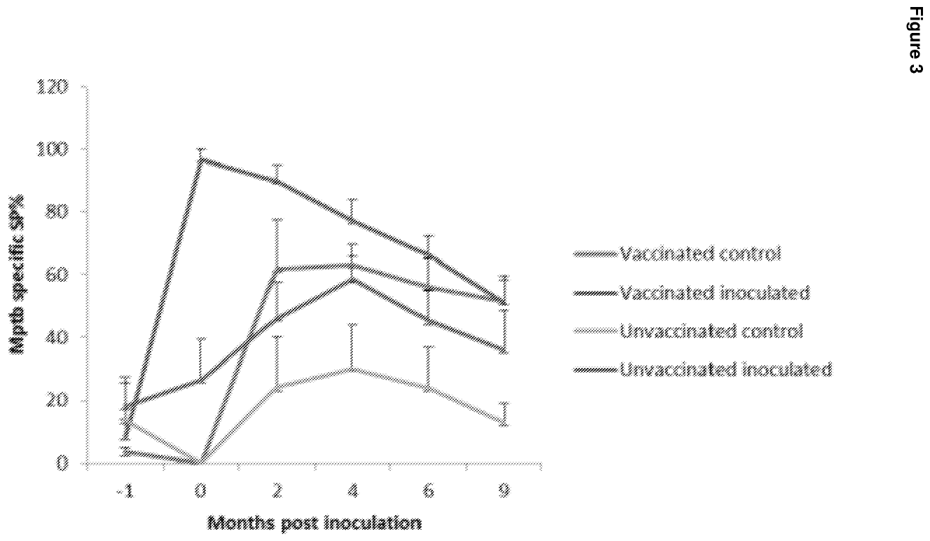

FIG. 4: Severity of histological lesions in gut tissues of Mptb inoculated animals, following vaccination with novel vaccine CV1 (heat killed Mptb in mineral oil CAS no: 8042-47-5).

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described.

It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

The present invention is based on the recognition by the inventors, that while the adjuvant portion of a vaccine plays an important role in its efficacy, it can also be responsible for the adverse effects (such as injection site lesions) resulting from vaccination.

The present inventors have developed novel vaccine compositions which may provide an improvement over the Mycobacterium avium subspecies paratuberculosis vaccine compositions of the prior art. In particular, the inventors have developed vaccine compositions which provide suitable efficacy for minimising clinical signs of Mycobacterium avium subspecies paratuberculosis infection, and reducing transmission of the pathogen between animals in a population, but which provide for greater safety in the form of reduced numbers of lesions at the site of injection.

Importantly the inventors have demonstrated a significant advantage of the presently claimed formulations, over the leading commercially available vaccine. Without wishing to be bound by theory, the inventors believe that the improved outcome, in the form of reduced numbers and sizes of lesions at the site of injection, derives from the specific mineral oil adjuvant selected. In particular, while commercially available vaccine formulations for immunising against Mptb also comprise mineral oils, the mineral oils identified by the present inventors, provide for a superior outcome in the form of a vaccine which is safer, both for the user and the animal recipient.

As used herein, the terms “vaccine” or “immune stimulating composition” refers to an antigenic preparation used to produce immunity to a disease, in order to prevent or ameliorate the effects of infection. The vaccine compositions of the present invention are therefore used to prevent or ameliorate the effects of infection with the organism Mycobacterium avium subspecies paratuberculosis.

The skilled person will be familiar with various abbreviations or synonyms for Mycobacterium avium subspecies paratuberculosis, including for example, the abbreviations M. paratuberculosis, M. avium ssp. paratuberculosis, MAP or Mptb, which are used herein interchangeably. Mycobacterium avium subspecies paratuberculosis is the causative agent of Johne's disease in cattle and other ruminants. In the context of bovine Johne's disease, this may be abbreviated to BJD. Johne's disease in sheep may be referred to as OJD (for ovine Johne's disease).

Typically, the subject in which the immune response is required is a ruminant. As used herein, the term ‘ruminant’ includes cattle, sheep, goats, camelids, deer, bison, buffalo and related species including wild and zoo animals. In a particularly preferred embodiment of the present invention, the vaccine compositions described herein are for use in eliciting an immune response in a bovine (cattle) or ovine (sheep) or caprine (goat) subject.

In certain embodiments, a single vaccine composition as described herein may be useful for eliciting an immune response in more than one species of subject (i.e., it may be useful for eliciting immune responses in both bovine and ovine subjects).

Vaccines are typically prepared using a combination of an immunologically effective amount of the immunogen together with an adjuvant effective for enhancing the immune response of the vaccinated subject against the immunogen. The process of distributing and administrating vaccines is referred to as “vaccination”.

The term “immunization” refers to the process by which a subject is exposed to a material that is designed to stimulate its immune system against that material. The material is known as an “immunizing agent” or “immunogen” or in certain contexts, “antigen”. When the immunizing agent is administered to a subject, the subject develops an immune response, which can be used for prevention and treatment against Johne's disease.

As used herein, the term “adjuvant” refers to a composition that enhances the effectiveness of the immunogen (i.e., potentiates the immune response of the individual to the immunogen). Adjuvants provide enhanced immune response even after administration of only a single dose of the vaccine.

There are many examples of adjuvants known in the art. Generally, an adjuvant for use in accordance with the present invention is a vaccine ingredient that stimulates the immune response of a subject in a non-specific manner. Many different adjuvants are known in the art. Examples of adjuvants are: Freund's Complete and -Incomplete adjuvant, vitamin E, non-ionic block polymers and polyamines such as dextran sulphate, carbopol and pyran, aluminium compounds such as Alum-phosphate or Alum-hydroxide, Saponin.

The present inventors have identified adjuvants which can be used in accordance with the present invention, and which have a greater safety profile than other adjuvants in common usage. Specifically, the inventors have found that the refined mineral oils as described herein, are not only useful in the preparation of vaccine compositions by providing for a suitable potentiation of immune response in an individual requiring immunisation, but which also are useful in that vaccines containing these adjuvants typically provide a greater safety profile in the form of reduced likelihood and severity of lesions at the site of injection, as compared with the currently available commercial vaccine formulations.

As such, in preferred embodiments of the present invention, the adjuvant for use in the compositions of the present invention is a refined mineral oil.

As used herein, the term ‘mineral oil’ (and synonyms such as paraffin oil or white mineral oil) refers to various colourless, odourless, light mixtures of higher alkanes from a mineral source, including distillates of petroleum.

In a particularly preferred embodiment of the present invention, the adjuvant for use in potentiating an immune response to Mycobacterium avium subspecies paratuberculosis, comprises a refined mineral oil selected from the group consisting of oils having the following identifiers: CAS no: 8042-47-5 (also known as EC no: 232-455-8); EC no: 932-078-5, EC no: 934-954-2, or EC no: 934-965-3. In a preferred embodiment, the mineral oil adjuvants described herein, for example, as defined by CAS no: 8042-47-5 or EC no: 232-455-8, EC no: 932-078-5, EC no: 934-954-2 or EC no: 934-956-3, is the sole adjuvant included in the vaccine or immune stimulating composition.

The mineral oil identified by CAS registry number 8042-47-5 is also known by EC number 232-455-8 or by the term “white mineral oil, petroleum”. This mineral oil can be obtained from a wide variety of commercial sources as: Britol white mineral oil (Sonneborn Inc, NJ, USA), Drakeol mineral oil (Penreco, PA, USA) or Marcol 52 (ESSO). The skilled person will understand from the teaching of the present disclosure that any preparation identified as containing a mineral oil identified CAS registry number 8042-47-5, especially those preparations indicated as being suitable for injection, can be used in the compositions of the present invention.

Another refined mineral oil which may be used in accordance with the present invention is a mixture of hydrocarbons described in accordance with the European REACH or IUPAC nomenclature, as “Hydrocarbons, C-13-C23, n-alkanes, isoalkanes, cyclics, <0.03% aromatics”. This mixture may also be referred to by its EC number 932-078-5. This mineral oil is also commonly referred to as having the related CAS registry numbers 64742-46-7 [R] or 64742-47-8 [R] and can be obtained from a variety of commercial sources as: Aqualane, Eolane 160 or Hydroseal (Total Special Fluids, France).

Related mineral oils may also be used as adjuvants in the compositions of the present invention. For example, oils described by the European REACH as “Hydrocarbons, C-13-C16, n-alkanes, isoalkanes, cyclics, <0.03% aromatics” (having EC number 934-954-2), or “Hydrocarbons, C-15-C20, n-alkanes, isoalkanes, cyclics, <0.03% aromatics” (having EC number 934-956-3) may also be used. These mineral oils can be purchased commercially as Berylane 230 and Berylane 250, respectively, or as Eolane 100, or Eolane 130, respectively.

The mineral oil adjuvant is preferably one which is liquid at 4° C. and has a viscosity lower than 100 mPas at 25° C. It preferably has a density at 20° C. of about 815 to 870 kg/m³, more preferably about 820 to 860 kg/m³. The dynamic viscosity of the oil at 25° C. is preferably about 5 to 300 mPas, preferably 20 to 250 mPas, more preferably about 30 to 200 mPas.

The present invention also contemplates the use of more than one adjuvant in the vaccine compositions described herein. This may have the effect of further potentiating the immune response to the immunogen. For example, a combination of different mineral oils may be used to potentiate the immune response to the immunogen. Moreover, a mineral oil may be combined with additional compounds for forming the adjuvant component of the vaccine compositions. Examples, of suitable compounds for combining with mineral oil adjuvants include saponins, which are surface-active glycosidic compounds. The saponin may be combined with the immunogen prior to mixing with the mineral oil adjuvant. Alternatively, the mineral oil and saponin may be combined prior to mixing with the immunogen. Examples of commercially available saponins include Quil A™ (Brenntag), Q-vac™ (Biolang), VaxSap™ (Desert King), and Abisco100™ (Isconova). A saponin adjuvant is preferably comprised in the vaccine according to the invention, at a level between 10 and 10,000 μg/ml, more preferably between 50 and 5000 μg/ml, even more preferably 20 between 100 and 1000 μg/ml.

The skilled person will appreciate that in the context of the present invention, the immunogen may be in the form of any number of different antigens to which the recipient can develop an immune response.

For example, the immunogen can be in the form of any of the following:
a plurality of whole killed Mycobacterium avium subspecies paratuberculosis organisms,
a plurality of live attenuated Mycobacterium avium subspecies paratuberculosis organisms,
a cell lysate formed from a plurality of Mycobacterium avium subspecies paratuberculosis organisms,
one or more peptides or polypeptides having the sequence of an Mycobacterium avium subspecies paratuberculosis protein.

In any embodiment of the invention, where the immunogen is in the form of whole killed Mycobacterium avium subspecies paratuberculosis organisms, the organisms may be killed by heat treatment. Typically, heat killing of whole cells can be accomplished by incubating the cells at approximately 70° C. for 2 hours and then confirmed by liquid culture (i.e., where killing is confirmed if there is no cell growth in the liquid culture following standard culturing conditions).

The generation of live attenuated strains will be within the skill set of the person skilled in the art. An attenuated strain of Mycobacteria is one which has been genetically modified so as to reduce or remove its ability to cause active disease (i.e., having reduced virulence) but which can be recognised as a source of antigens against which an immune response can be generated. Attenuated strains of Mycobacterium avium subspecies paratuberculosis are known in the art (for example, as described in Settles et al., (2014) Vaccine, 11: 32: 2062-9) however, it will be appreciated that any attenuated strain of MAP may be used in the vaccine compositions of the present invention.

The vaccine compositions of the present invention may also comprise an immunogenic polypeptide as the relevant source of immunogen. The immunogenic polypeptide may be any peptide from Mycobacterium avium subspecies paratuberculosis to which the subject receiving the peptide may develop an immune response. Examples of immunogenic polypeptides from Mycobacterium avium subspecies paratuberculosis are described in the art, for example, any polypeptide corresponding to the gcpE, pstA, kdpC, papA2, impA, umaA1, fabG2_2, aceAB, mbtH2, IpqP, mapO834c, cspB, HpN, or map1634 proteins of M. paratuberculosis, or homologs of these proteins (as described in WO2007/075308 or WO2007/017635, the entire contents of which are hereby incorporated by reference in their entirety. In certain embodiments, the immunogenic polypeptide may correspond to a protein that is secreted by Mycobacteria. Thus, while in certain circumstances the immunogenic polypeptide may include a recombinantly produced protein that is then purified from a cell culture, it will be appreciated that it is also possible to prepare a suitable immunogen from the supernatant of cultured Mycobacteria (such that the supernatant contains the secreted protein that forms the immunogen). It will be within the purview of the person skilled in the art to select and prepare suitable preparations of immunogenic polypeptides for use of the vaccine compositions and methods described herein.

The present invention also contemplates the use of a whole cell lysate obtained from Mycobacterium avium subspecies paratuberculosis for use as an immunogen. A lysate formed from a plurality of cultured Mycobacteria can be produced by physical (French press, sonifier), or by chemical (detergents, chaotropic agents) means. The suspension may be further purified, or be concentrated, e.g. by centrifugation or filtration.

The skilled person will appreciate that it is possible to adapt the vaccine or immune stimulating composition to be directed to more than one strain of Mycobacterium avium subspecies paratuberculosis (a so-called multi-valent vaccine, ie. a vaccine providing protection against a number of different MAP strains by incorporating a number of different MAP antigens). This may be desirable, for example, where it is necessary to develop vaccines targeted to specific strains of Mycobacterium avium subspecies paratuberculosis prevalent in a given geographical region. Further, it is known that various strains of Mycobacterium avium subspecies paratuberculosis are more likely than others to infect given cattle. As such, it will be within the purview of the skilled person to be able to determine which strain of Mycobacterium avium subspecies paratuberculosis to utilise in obtaining the immunogen, depending on the intended use of the vaccine composition.

In any embodiment of the present invention, the immunogen used in the vaccine composition may be derived from Mycobacterium avium subspecies paratuberculosis organisms of strain 316F. Alternatively, the immunogen may be derived from Mycobacterium avium subspecies paratuberculosis strain Telford or strain CM 00/416 (common sheep strains), K10 (a common cattle strain, also referred to as BAA-968).

Vaccine formulations will contain a “therapeutically effective amount” of the immunogen, that is, an amount capable of eliciting an immune response in a subject to which the composition is administered. In the treatment and prevention of Johne's disease, for example, a “therapeutically effective amount” would preferably be an amount that enhances resistance of the vaccinated subject to new infection and/or reduces the clinical severity of the disease. Such protection will be demonstrated by either a reduction or lack of signs normally displayed by a subject infected with Johne's disease, a quicker recovery time and/or a lowered count of M. paratuberculosis bacteria. Other examples of protection provided by the vaccine formulations disclosed herein include: reduction or prevention of M. paratuberculosis shedding, reduction or prevention of gross pathological signs consistent with Johne's disease, reduction or prevention of histopathological lesions consistent with M. paratuberculosis, reduction or prevention of M. paratuberculosis invasion of intestinal and/or other body tissues, reduction or prevention of M. paratuberculosis shedding in milk, reduction or prevention of intrauterine infection of foetus, reduction or prevention of clinical signs such as weight loss and diarrhoea.

The skilled person will be familiar with techniques for formulating and preparing the vaccine compositions of the present invention. In their simplest form, the compositions of the present invention can be formed simply by mixing the relevant immunogen with the relevant adjuvant in the appropriate quantities, using conventional methods for vaccine preparation.

In certain embodiments, the immunogen is provided in a substantially aqueous phase (for example, if the immunogen is a sample of killed whole cell bacteria provided in PBS buffer or the like). In these circumstances, it may be desirable to provide a suspension or an emulsion of the immunogen and the adjuvant for administration to a subject in need thereof.

In certain embodiments, the immunogen may be provided in the lipid phase (ie. non-aqueous phase) of the vaccine formulation.

The vaccine composition may be an injectable emulsion of the “water in oil” type and preferably has a viscosity of about 200 mPas or less, more preferably about 100 mPas to about 150 mPas.

In certain embodiments, the vaccine may be in the form of an oil-in-water type emulsion.

In order to provide the immunogen and adjuvant as an emulsion, the compositions of the present invention may further comprise an emulsifier. Examples of emulsifiers which can be used in the compositions of the present invention include any of a wide variety of emulsifiers that are suitable for emulsifying mixtures of water and oil. The emulsifier may be of animal or non-animal origin, including from plant origin. Alternatively, the emulsifier may be chemically synthesized.

Examples of suitable emulsifiers include mannide monooleates, polyoxyethylene ethers (or octoxynols) such as lauryl, cetyl, oleyl, stearyl, and tridecyl polyoxyethylene ethers; polyoxyethylene sorbitan-fatty acid esters (commonly sold under the trade name TWEEN), such as polyxoethylene 20 sorbitan monolaurate (TWEEN 20; also called Polyethylene glycol sorbitan monolaurate or Polyoxyethylenesorbitan monolaurate), polyoxyethylene (60) sorbitan monolaurate (TWEEN 60); polyoxyethylene ethers such as TRITON X-100, X-102, X-165 and X-305; fatty acid diethanolamides such as isostearic acid DEA, lauric acid DEA, capric acid DEA, linoleic acid DEA, myristic acid DEa, oleic acid DEA, and stearic acid DEA; fatty acid monoethanolamides such as coconut fatty acid monethanolamide; fatty acid monisopropanolamides such as oleic acid monoisopropanolamide and lauric monoisopropanolamide; alkyl amine oxides such as N-cocodimethylamine oxide, N-lauryl dimethylamine oxide, N-myristyl dimethylalmine oxide, and N-stearyl dimethylamine oxide; N-acyl amine oxides such as N-cocoamidopropyl dimethylamine oxide and N-tallowamidopropyl dimethylamine oxide; and N-alkoxyalkyl amine oxides such as bid (2-hydroethyl) C12-C15 alkoxy-propylamine oxide.

In any embodiment of the present invention, the emulsifier is a mannide monooleate (also called dianhydro-D-mannitol monooleate; dianhydromannitol monooleate). Preferred examples of a mannide monooleate include those sold under the trade name Arlacel (having CAS registry number 25339-93-9 or CAS registry number 9049-98-3). The mannitol oleate emulsifier is preferably an anhydromannitol ether octadecanoate. Preferred emulsifiers have a viscosity at 25° C. of about 300 to 400 cP, more preferably about 340 to about 360 cP, particularly preferred embodiments are those in which the emulsifier has a viscosity of about 350 cP. The emulsifier preferably has a specific gravity at 20° C. of about 0.8 to 1.0, more preferably of about 0.95 to about 0.99, particularly suitable are those with a specific gravity at 20° C. of about 0.97. Particularly preferred emulsifiers are those with a refractive index at 25° C. of about 1.4 to 1.5, more preferably of about 1.47 to 1.48, particularly those with a refractive index at 25° C. of about 1.4748 to 1.4758.

The amount of the emulsifier used will be sufficient to emulsify the aqueous component (i.e., the immunogen) with the oily component (the adjuvant for potentiating the immune response to the immunogen). The skilled person will be familiar with methods for determining the appropriate amount of emulsifier to include with the immunogen and adjuvant for use in the compositions of the present invention.

Typically, the adjuvant, in the form of a mineral oil is preferably between about 50% and about 70% by weight of the emulsion more preferably between about 53% and about 63% by weight of the emulsion.

Generally, where mannitol oleate emulsifier is used, it is preferably between about 2% and about 10% by volume of the emulsion, more preferably between about 3% and about 7%.

It will be apparent to a person of skill in the art that the proportion of oily adjuvant to aqueous phase included in the emulsion can be adjusted to optimise the efficacy of the vaccine to elicit an immune response.

In formulating the compositions of the present invention, the emulsifier and adjuvant may be combined prior to mixing with the immunogen. In other words, where the immunogen is provided in an aqueous phase, the adjuvant and emulsifier will be mixed first, and then combined with the immunogen to form a water-in-oil emulsion. The total water-to-oil ratio will preferably be between 30:70 and 70:30, and more preferably around 50:50, to have an injectable emulsion with acceptable viscosity.

Compositions comprising a ready-made mixture of emulsifier and a mineral oil can also be purchased commercially and used in accordance with the present invention. In this case, it will be evident to the skilled person that the “adjuvant for potentiating the immune response to the immunogen” and the emulsifier can be provided together in a single mixture, for combining with the immunogen to form the vaccine composition of the present invention. Examples of commercially available compositions comprising the potentiating compound and an emulsifier include the Montanide series of adjuvants sold by Seppic SA (75 Quai D-Orsay, 75007 Paris) including Montanide ISA 50 V2, Montanide ISA 201 VG, Montanide ISA 61 VG and Montanide ISA 71 VG. These mixtures comprise a high grade injectable mineral oil and an emulsifier obtained from mannitol and purified oleic acid of vegetable origin.

The skilled person will be familiar with techniques in the art for preparing a vaccine composition comprising the components as recited herein (i.e., an immunogen in aqueous media, a mixture of hydrocarbons for potentiating the immune response and an emulsifier). For example, the vaccine composition may be prepared by mixing the aqueous medium containing the immunogen into an equal volume of potentiating compound/emulsifier mixture, at room temperature, under vigorous mixing. In certain embodiments, it may be necessary to use high shear mixing to ensure preparation of a stable, homogenous and efficient vaccine composition. Methods for optimising the mixing of the components of the vaccine composition will be within the skill set of the person skilled in the art.

The compositions described herein may also include diluents, excipients and carriers enabling administration of the composition, as known in the art. A “pharmaceutically acceptable carrier” means any conventional pharmaceutically acceptable carrier, vehicle, or excipient that is used in the art for production and administration of vaccines. Pharmaceutically acceptable carriers are typically non-toxic, inert, solid or liquid carriers.

Prior to administration to subjects as a vaccine, the vaccines described herein are tested according to methods that are well-known to those of skill in the art. For example, tests for toxicity, virulence, safety, etc. are carried out in suitable animal models, e.g. in cattle, sheep, etc. The ability of the vaccine preparations to elicit an immune response is likewise typically tested in suitable animal models, e.g. cattle, sheep. In addition, protection studies involving vaccination, boosting, and subsequent challenge with live bacteria may be carried out using suitable animal models.

The skilled person will be familiar with determining the appropriate dose of immunogen to administer. For example, where the immunogen is in the form of attenuated or killed whole cell Mycobacterium, the appropriate dose of immunogen to administer to an animal requiring immunisation will be approximately 1×10⁷-1×10¹⁰ cells/dose. In a preferred embodiment, the dose administered is between 1×10⁹-1×10¹⁰ cells/dose.

Moreover, where the skilled person will appreciate that the dose may also depend on the strain for which the vaccine composition is being used. For example, where the vaccine composition contains immunogen derived from more than one strain of Mycobacterium, the skilled person will be able to adjust the dosing accordingly, so as to maximise the amount of immunogen received by the animal.

The vaccine compositions of the present invention are particularly useful for reducing the severity of one or more signs of infection with Mycobacterium avium subspecies paratuberculosis. In cattle, the main signs of Johne's disease include diarrhoea and wasting. Initial signs may be subtle and may be limited to weight loss, decreased milk production or roughening of the hair coat. The diarrhoea may be intermittent and typically without blood or mucous or epithelial debris. Several weeks after the onset of diarrhoea, a soft swelling may occur under the jaw of infected subjects (known as ‘bottle jaw’ or ‘intermandibular oedema’). This sign results from loss of protein from the bloodstream into the digestive tract.

The skilled person will be familiar with methods for determining the clinical significance of any infection with MAP. For example, tests for determining the extent of faecal shedding and tissue burden of MAP are described in Kawaji et al., (2014) J. Vet. Med. Sci., 76: 65-72, Whittington and Sergeant (2001) Australian Veterinary Journal 79: 267-78; Whittington (2010) in Behr and Collins (eds) Paratuberculosis Organism, Disease, Control. CABI, Wallingford; and Collins (2011) Veterinary Clinics of North America; Food Animal Practice, 27: 581-591, the entire contents of which are herein incorporated in their entirety.

Vaccines can be administered prior to infection, as a preventative measure against Johne's disease. Alternatively, vaccines can be administered after the subject already has become infected with Mycobacterium (for example, to reduce the severity of the infection, reduce or ameliorate clinical signs of infection, or reduce transmissibility of infection to other subjects). Vaccines given after exposure to Mycobacteria may be able to attenuate the disease, triggering a superior immune response than the natural infection itself.

The vaccines provided by this invention may be administered subcutaneously, intramuscularly, intradermally, or into an organ. The chosen route of administration will depend on the vaccine composition and the disease status of subjects. Relevant considerations include the types of immune cells to be activated, the time which the antigen is exposed to the immune system and the immunization schedule. Although many vaccines are administered consecutively within a short period, spreading the immunizations over a longer time may maintain effective clinical and immunological responses.

To immunize a subject, the vaccine is preferably administered parenterally, usually by subcutaneous injection. Other modes of administration, however, such as intramuscular, intraperitoneal and intravenous injection, are also acceptable. The quantity to be administered depends on the subject to be treated, the capacity of the subject's immune system to synthesize antibodies, and the degree of protection desired. Effective dosages can be readily established by one of ordinary skill in the art through routine trials establishing dose response curves. The subject is immunized by administration of the vaccine in at least one dose, and preferably two to four doses. Moreover, the subject may be administered as many doses as is required to maintain a state of immunity to infection.

The various stages of the manufacturing process will be monitored by adequate tests, for instance by immunological tests for the quality and quantity of the antigens; by microbiological tests for inactivation, sterility, and absence of extraneous agents; and ultimately by studies in animals for confirming vaccine efficacy and safety. All these are well known to a skilled person. General techniques and considerations that apply to the preparation of vaccines are well known in the art and are described for instance in governmental regulations (Pharmacopoeia) and in well-known handbooks.

The invention is also directed to a kit for vaccination against Johne's disease. The kit may include one or more of a sample that includes an immunogen, and an adjuvant and a delivery device (for example, for an injection). The kit may include instructions for using the kit.

### EXAMPLES

### Example 1—Tissue Reactivity of Vaccines Containing Different Adjuvants

Methods

Animals

Ninety Merino wethers aged 24-36 months were sourced from a flock in Armidale, New South Wales, an area that has no prior history of JD. Absence of JD was confirmed through repeated whole flock faecal tests and antibody enzyme linked immunosorbent assays (ELISA) (Begg et al. 2010). The animals were moved to a JD-free farm at the University of Sydney Camden and maintained under conventional Australian sheep farming conditions by grazing on open pasture.

Ethics

All animal experiments were conducted with the approval of the University of Sydney Animal Ethics Committee.

Treatment Groups

Sheep were drafted and systematically randomised into 18 groups with five sheep in each group. The first eight groups of animals were allocated for a single dose of the novel vaccines including one with no adjuvant (Table 1). The subsequent eight groups of animals were allocated a double dose schedule of the novel vaccines. One group was a “vaccine positive” control by using a commercially available vaccine in a single dose as recommended by the manufacturer. A negative control group was not vaccinated. The adjuvant and antigen components are listed in Table 1.

TABLE 1
Vaccine formulations tested

| Group | Vaccine | Adjuvant | Antigen |
|---|---|---|---|
| 1 | Adjuvant 1 | White mineral oil | Heat killed Mptb 1 × |
| | | CAS no 8042-47-5 | 10⁸/dose |
| 2 | Adjuvant 2 | Mineral oil EC no | Heat killed Mptb 1 × |
| | | 932-078-5 | 10⁸/dose |
| 3 | Adjuvant 3 | Mineral oil EC no | Heat killed Mptb 1 × |
| | | 932-078-5 | 10⁸/dose |
| 4 | Adjuvant 4 | Mineral oil EC no | Heat killed Mptb 1 × |
| | | 932-078-5 | 10⁸/dose |
| 5 | Adjuvant 5 | Mineral oil EC no | Heat killed Mptb 1 × |
| | | 932-078-5 | 10⁸/dose |
| | Adjuvant 6 | Gel dispersion of | Heat killed Mptb 1 × |
| | | sodium polyacrylate | 10⁸/dose |
| 7 | No adjuvant | Phosphate buffered | Heat killed Mptb 1 × |
| | | saline | 10⁸/dose |
| 8 | Positive vaccine | As supplied by | Killed Mptb as supplied |
| | control | manufacturer | in the vaccine by the |
| | (commercially | | manufacturer |
| | available vaccine) | | |
| 9 | Negative control | None | None |
| | (no vaccine) | | |

Adjuvants

Various adjuvants and water:oil emsulsions of adjuvants and immunogens were tested. The adjuvants comprises mineral oils as defined either by CAS no: 8042-47-5 (white mineral oil), by EC no: 932-078-5 (hydrocarbons, C-13-23, n-alkanes, isoalkanes, cyclics, <0.03% aromatics), or a gel dispersion of sodium polyacrylate. Phosphate buffered saline (PBS) was used as a control (i.e., a “no adjuvant” control). A commercially available Mptb vaccine was used as a positive vaccine control.

Vaccines

Eight vaccine formulations were used in this study. The commercially available vaccine comprised killed Mptb (Strain 316f) cells in a mineral oil adjuvant as prepared by the manufacturer. A single dose of the novel formulations contained approximately 1×10⁸ organisms of Mptb (Telford strain heat killed at 70° C. for 2 hours). Mptb inactivation was confirmed by liquid culture (Whittington et al. 1999).

Antigens were emulsified with adjuvant under aseptic conditions. All novel vaccine formulations were tested for sterility by aerobic culture on sheep blood agar incubated ay 37° C. for 48 hours, prior to use.

Vaccination

The vaccines were administered via subcutaneous injection high on the neck as a 1 mL dose, behind the ear. All vaccinations were given on the right side of the neck. At one month post primary administration, groups requiring a booster dose were given a second dose of the same vaccine formulation. The commercially available vaccine was administered as a single dose according to the manufacturer's instructions on the right side of the neck.

Collection of Blood Samples

Serum samples (9 mL) were collected from all animals immediately before vaccination and at 2, 3, 4, 5, 6, 7, 8, 10, 14, 18, 22 and 26 weeks post primary vaccination. Blood samples for the IFNγ assay were collected at pre-vaccination and then monthly for 6 months by jugular venipuncture into vacuum collection tubes (Vacuette). Serum samples were stored at −200° C. until required while heparinised blood was held at room temperature (≤5 hr) prior to stimulation with antigens for the IFNγ assay.

Assessment of Injection Site Lesions

The site of injection was monitored weekly until 10 weeks post vaccination and then monthly until 6 months post vaccination. The area around the injections site was palpated for the presence of any adverse reactions such as a swelling, lumps, open lesions or abscess formation. Injection site lesions were defined as having a diameter greater than 0.5 cm, measured in one axis. Smaller lesions were detected by palpation, but not frequently or consistently, and were therefore not included in the data set. Injection site lesion data are presented as the percentage of animals with lesions in each treatment aggregated across all the observations.

Serological Assay to Measure Antibodies Specific to Mptb Vaccination

An indirect ELISA was employed to detect Mptb specific antibody in serum (Gurung et al. 2013). Serum samples (10 μL) were thawed to room temperature and adsorbed against Mycobacterium phlei (1.3 mg/mL) diluted in 0.1% w/w foetal calf serum (FCS) in phosphate buffered saline (PBS) (Amresco) Tween 20 (0.05%, v/v) (PBST), (990 μL). The samples were mixed by end-to-end rotation whilst being incubated at 4° C. overnight.

Mptb 316v antigen (EMAI, NSW, Australia) was diluted in carbonate buffer (pH 9.6) to a concentration of 2 μg/mL. ELISA plates (Nunc, MaxiSorp) were coated with 50 μL of diluted antigen in each well. The plates were incubated overnight at 4° C. Plates were machine washed five times (Tecan, Austria) using wash buffer (Reverse Osmosis (RO) with 0.05% w/w Tween 20) prior to the addition of 100 μl of 1% FCS in PBST to all wells, then incubated at room temperature for 30 minutes.

The M. phlei adsorbed sera were centrifuged at 2500×g for 10 minutes at 4° C. Plates were again washed (5×) using wash buffer prior to the addition of 50 μl of diluted sera to appropriate wells. This was followed by a 1 hour incubation at room temperature. The secondary antibody (HRP-labelled monoclonal mouse anti-sheep IgG clone GT34, Sigma) (50 μL) was added at a concentration of 0.5 μg/mL diluted in PBS, incubated for 1 hour at room temperature and then washed five times. TMB substrate (3, 3′ 5, 5′-tetramethylbenzidine and hydrogen peroxide, 100 μL) was added to each well and the plates were incubated in the dark for 20 minutes. The reaction was stopped using 2 M sulphuric acid (50 μL). The optical density (OD) was measured in an ELISA plate reader (Multiskan Ascent, Thermo Electric Corporation) at 450 nm. Results were expressed as the mean optical density signal from two replicates.

IFN Gamma (IFNγ) Assay

Heparinised blood (0.5 mL) was stimulated in a 48-well plate with 0.5 mL of mycobacterial purified protein derivative (PPD) antigen (Prionics) at 20 μg/mL. The negative control for each sample consisted of blood with 0.5 mL of culture medium while the positive control had 0.5 mL of media with pokeweed mitogen (Sigma) added at 10 μg/mL. After 48 hr incubation at 37° C. in air supplemented with 5% CO₂, the plasma supernatant was collected and stored at −20° C. The ELISA was carried out as described by Begg et al 2010 (Begg, de Silva et al. 2010).

Results

Injection Site Lesions

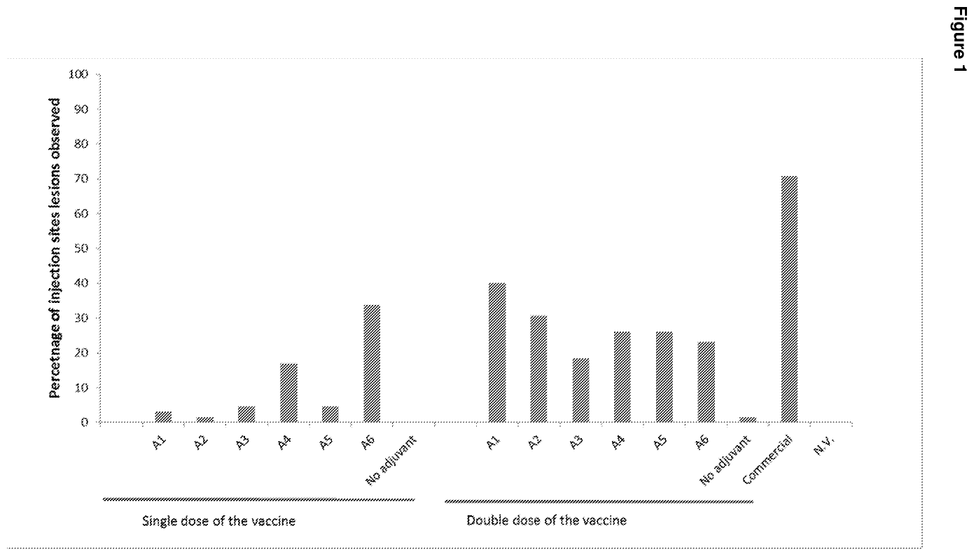

Sheep with the commercially available vaccine had a greater probability of having an injection site lesion than did sheep given the other novel Mptb vaccine formulations (Tables 2 and 3). Sheep receiving two doses of the modified Mptb vaccines had a greater probability of having an injection site lesion present than did the animals that received only one dose (25% compared to 7%). When the data for the single and double dose vaccinated sheep to the novel Mptb vaccines were combined, the probability that an animal may have an injection site lesion was not significantly different, although there was a trend that double dose vaccinated sheep had more injection site lesion observations. The overall proportion of animals that had a lesion identified for each treatment group is shown in FIG. 1. Compared to the commercially available vaccine, and for a single dose administration (which is the preferred method of immunisation), the novel formulations tested resulted in fewer animals with lesions, the size of the lesions was reduced. Even when 2 doses were administered (ie, intending to achieve an adverse reaction), the novel formulations tested herein resulted in smaller, less persistent lesions than obtained with the commercial vaccine.

TABLE 2
Injection site lesions in sheep given one dose of the vaccine
formulations of killed Mptb and different adjuvants

| | No | No lesion | | Mean weeks | Mean weeks | Mean weeks |
|---|---|---|---|---|---|---|
| | animals | observations | Mean | to first | to last | between first |
| | with lesions | in treatment | lesion | recorded | recorded | and last lesion |
| Vaccine | observed | group | size (cm) | lesion | lesion | observation |
| 1 | 1 | 2 | 0.9 | 1 | 6 | 5 |
| 2 | 1 | 1 | 0.8 | 3 | 1 | 1 |
| 3 | 1 | 3 | 1.1 | 2 | 26 | 24 |
| 4 | 1 | 11 | 1.3 | 1 | 26 | 25 |
| 5 | 1 | 3 | 2.0 | 8 | 14 | 6 |
| 6 | 2 | 22 | 1.4 | 1.5 | 26 | 24.5 |
| 7 | 0 | 0 | 0.0 | — | — | — |
| (no adjuvant) | | | | | | |
| 8 | 5 | 46 | 2.2 | 2.2 | 26 | 23.8 |
| Commercial | | | | | | |
| vaccine | | | | | | |
| 9 | 0 | 0 | 0.0 | — | — | — |
| (unvaccinated) | | | | | | |

TABLE 3
Injection site lesions in sheep given two doses of the formulations
of killed Mptb and different adjuvants

| | No | No lesion | | Mean weeks | Mean weeks | Mean weeks |
|---|---|---|---|---|---|---|
| | animals | observations | Mean | to first | to last | between first |
| | with lesions | in treatment | lesion | recorded | recorded | and last lesion |
| Vaccine | observed | group | size (cm) | lesion | lesion | observation |
| 1 | 4 | 26 | 1.9 | 2.5 | 19.3 | 17 |
| 2 | 4 | 20 | 2.1 | 5.75 | 21.5 | 15.75 |
| 3 | 3 | 12 | 2.2 | 6.0 | 16.7 | 10.7 |
| 4 | 3 | 17 | 2.0 | 5.7 | 20.0 | 14.7 |
| 5 | 5 | 17 | 1.5 | 9.6 | 21.2 | 12 |
| 6 | 4 | 15 | 1.3 | 3.75 | 11.25 | 8.25 |
| 7 | 1 | 1 | 1.2 | 3 | 3 | 1 |
| (no adjuvant) | | | | | | |
| 8 | 5 | 46 | 2.2 | 2.2 | 26 | 23.8 |
| Commercial | | | | | | |
| vaccine | | | | | | |
| 9 | 0 | 0 | 0.0 | — | — | — |
| (unvaccinated) | | | | | | |

There were also significant differences in the size of the lesions produced (Tables 2 and 3), with the commercially available vaccine producing larger lesions overall (P<0.05).

### CONCLUSIONS

The results demonstrate that the number and size of lesions in sheep vaccinated with commercially available vaccine was greater than when sheep were vaccinated with a vaccine containing an adjuvant comprised of refined mineral oils, (as defined by White mineral oil CAS no 8042-47-5 or Mineral oil EC no 932-078-5). Therefore, the use of either white mineral oil CAS no 8042-47-5 or of mineral oil EC no 932-078-5 as an adjuvant with heat-killed Mptb provides for a benefit over the commercially available vaccine in that it results in fewer lesion number and size upon vaccination.

### Example 2—Efficacy of Vaccine Formulations

Methods

Thirty calves were sourced from a herd in NSW, Australia, which was shown to be free of Johne's disease by on-farm monitoring of the infection status of their dams using antibody ELISA, faecal culture and IS900 PCR on the whole herd. All calves were shown to be free from detectable Mptb infection by faecal culture, antibody and whole blood IFN-γ ELISA prior to the study. The animals were managed under conventional Australian farming conditions by grazing pasture in open paddocks.

The calves aged 1.5 months were allocated into a two groups of 15 to be inoculated. One group was tagged and the second group was tagged and vaccinated with a single dose of the Cattle Vaccine (CV) 1 (Table 1).

One month later the animals from each treatment group were divided into 2 groups, one group of 5 Unexposed controls (n=5) and a group of Mptb inoculated (n=10) calves (Table 1). Control animals were housed separately from the inoculated animals, in paddocks where no Mptb infected livestock had been housed in the past. The 20 animals to be inoculated were dosed orally using the same schedule as described previously (Begg et al., 2010) but using a cattle strain of Mptb. The inoculation doses were 8.6×10⁸, 4.2×10⁹ and 8.6×10⁹ viable cells of the Mptb cattle strain.

TABLE 4
Treatment groups of cattle receiving single dose of cattle vaccine

| | | | Heat killed | | No cattle |
|---|---|---|---|---|---|
| | | | Mptb | | inoc- |
| Vaccine | | | per 1 ml | No | ulated |
| Treatment | | | dose | doses | (n° |
| Group | Antigen | Adjuvant | vaccine | (1 ml) | controls) |
| Un- | — | — | — | — | 10 (5) |
| vaccinated | | | | | |
| CV1 | HK Mptb | White mineral | 1 × 10⁹ | 1 | 10 (5) |
| | cattle strain | oil CAS no: | | | |
| | | 8042-47-5 | | | |

Vaccinations

Vaccination was with a 1 mL dose of the cattle vaccine (CV1) behind the right ear of each animal.

Ante-Mortem Sampling and Examinations

From each animal blood and faecal samples were collected at regular intervals (1-3 months). All animals were monitored regularly by visual inspection, greater than three times weekly.

Necropsy and Tissue Collection

All animals were culled at 9 months post inoculation. Euthanasia of the animals and tissue sampling were as described by Begg et al 2010 with minor modifications. The tissues collected from each animal for Mptb isolation and histology were the terminal ileum, middle jejunum, posterior and middle jejunal lymph nodes and a section of the liver and hepatic lymph node and prescapular lymph node. Sections were either frozen at −80° C. for Mptb isolation or placed in 10% buffered formalin.

Histopathology

Tissues stored in buffered formalin were embedded in paraffin, sectioned at 5 mm sections and stained with haematoxylin and eosin and the Ziehl-Neelsen stains. The sections of intestine were graded as a score 0, 1, 2, 3a, (Paucibacillary) 3b (Multibacillary), or 3c (Severe Paucibacillary) using established criteria (Perez et al., 1996). Granulomatous lesions observed in the lymph nodes were graded as 1 (mild focal), 2 (mild multifocal) or 3 (severe multifocal to diffuse). Each animal was classified based on the highest grade of lesion observed across all regions of the gut assessed.

Mptb Isolation

Culture of Mptb from faeces and tissues was done using liquid culture media M7H9C as described previously (Plain et al., 2015; Whittington et al., 2013).

Mptb Specific Antibody Detection

Mptb specific antibodies were measured using a commercially available kit (Institut Porquier from Idexx) following the manufacturer's instructions. The data are presented as S/P %, which was calculated as: (OD sample−OD negative control)/(OD positive control−OD negative control)×100.

Mptb and MAP2698c Specific IFN-γ Detection

The IFN-γ assay was carried out using whole blood cultured with Mptb-specificantigen (316 v) for 48 hours as previously described (Begg et al., 2009).

Results

All animals tolerated the vaccination well and there were no adverse reactions or injection site lesions observed in the vaccinated cattle.

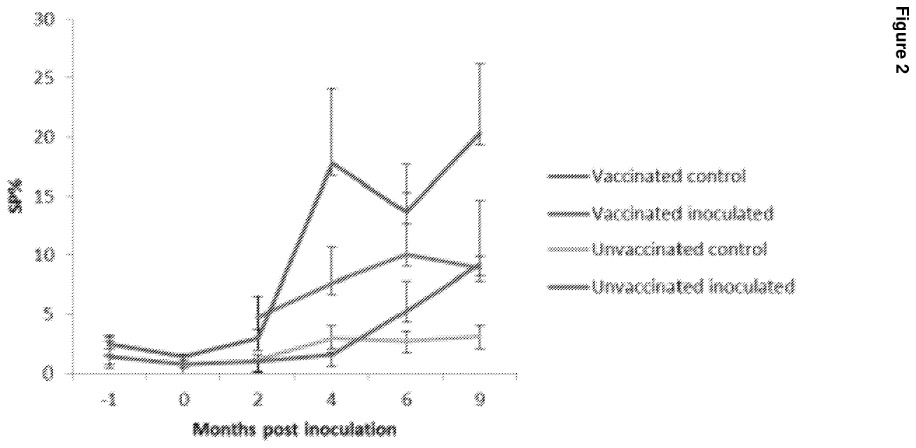

Vaccination with CV 1 induced a weak antibody response detectable in the vaccinated control (uninoculated) animals and also the vaccinated inoculated animals from 3 months post-vaccination (2 months post-inoculation). The CV1 vaccinated cattle that were inoculated with Mptb tended to have a stronger antibody response than all other groups (FIG. 2). The unvaccinated inoculated animals appear to have low levels of antibody that increased from 6-9 months postinoculation. Two animals, both inoculated, one vaccinated and the other not, had an SP % greater than 50, indicative of a positive test result in the IDEXX ELISA test used.

The IFN-γ responses were stronger after vaccination and the CV 1 vaccine induced a strong early response in association with exposure (FIG. 2). Vaccination alone produced an IFN-γ response that was greater than in unvaccinated control animals that were not exposed to Mptb.

Faecal shedding of Mptb was seen only in the inoculated animals. Of the shedding animals, 7 of the 8 animals were from the unvaccinated group. Mptb was seen in the faeces from 2-6 months post inoculation, with all the animals only showing intermittent shedding.

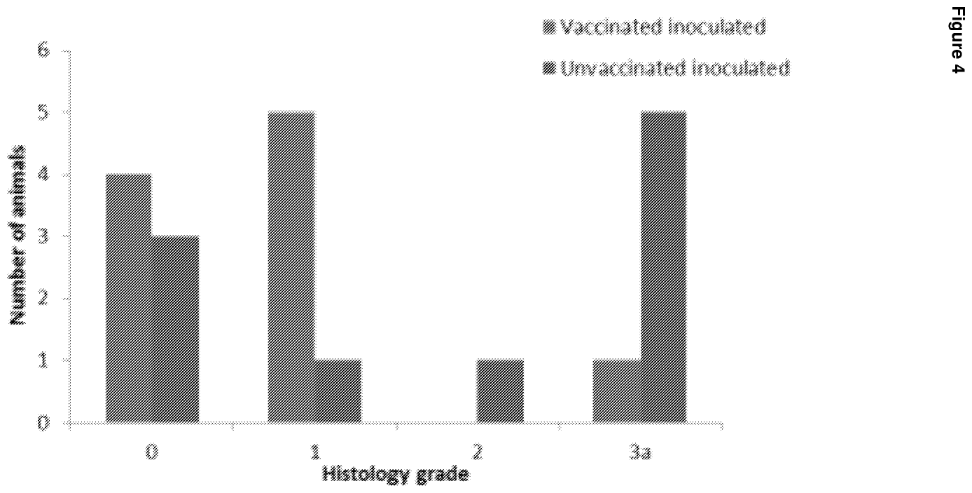

At necropsy, no gross lesions were observed in any of the animals. Histopathological lesions were observed from the Mtpb inoculated cattle. There were 6/10 unvaccinated animals and 1/10 vaccinated with lesions greater than score 1 at any location along the gut. The lesions were more severe in the unvaccinated animals with half the inoculated animals having score 3a lesions. Only one vaccinated Mptb inoculated animal had a 3a lesion. All 6 of the unvaccinated animals with lesions were found to be faecal shedding, but not the vaccinated animal with the score 3a lesion (see FIG. 4).

In both the vaccinated and unvaccinated groups, 9/10 Mptb inoculated animals had Mptb cultured from their tissues. Mptb was not cultured from any of the tissues of the animals not exposed to Mptb.

Discussion

In this trial, vaccination with prototype vaccine CV 1 was shown to give a positive benefit in terms of immunological markers and disease severity measures.

Cattle vaccinated with CV 1 had a markedly enhanced IFNγ response to exposure and also showed elevated antibody responses.

The histological and faecal shedding results indicate the animals vaccinated with CV1 had less severe infections and appeared to be protected from disease. The antibody levels of the inoculated animals were rising, with one animal test positive at 9 months post-inoculation.

The infection rate in this trial was 90%. This high number of tissue culture positives in both treatment groups is supportive of the argument that the vaccine is protective, because it is clear from this that there was an equivalent degree of tissue invasion/infection, but the vaccinated animals have reduced lesions associated with this.

The prototype CV 1 vaccine applied in this trial displays the traits of a desirable vaccine; protects against disease, reduces faecal shedding and does not cause injection site lesions.

It will be understood that the invention disclosed and defined in this specification extends to all alternative combinations of two or more of the individual features mentioned or evident from the text or drawings. All of these different combinations constitute various alternative aspects of the invention.

### REFERENCES

Begg, D. J., de Silva, K., Bosward, K., Di Fiore, L., Taylor, D. L., Jungersen, G., Whittington, R. J., 2009. Enzyme-linked immunospot: an alternative method for the detection of interferon gamma in Johne's disease. J Vet Diagn Invest 21, 187-196.
Begg, D. J., de Silva, K., Di Fiore, L., Taylor, D. L., Bower, K., Zhong, L., Kawaji, S., Emery, D., Whittington, R. J., 2010. Experimental infection model for Johne's disease using a lyophilised, pure culture, seedstock of Mycobacterium avium subspecies paratuberculosis. Vet Microbiol 141, 301-311.
Plain, K. M., Waldron, A. M., Begg, D. J., de Silva, K., Purdie, A. C., Whittington, R. J., 2015. Efficient, validated method for detection of mycobacterial growth in liquid culture media by use of bead beating, magnetic-particle-based nucleic acid isolation, and quantitative PCR. J Clin Microbiol 53, 1121-1128.
Whittington, R. J., Whittington, A. M., Waldron, A., Begg, D. J., de Silva, K., Purdie, A. C., Plain, K. M., 2013. Development and validation of a liquid medium (M7H9C) for routine culture of Mycobacterium avium subsp. paratuberculosis to replace modified Bactec 12B medium. J Clin Microbiol 51, 3993-4000.

1. A vaccine or immune stimulating composition comprising:
an immunogen for providing an immune response to Mycobacterium avium subspecies paratuberculosis in a subject,
an adjuvant for potentiating the immune response to the immunogen,
thereby forming a vaccine or immune stimulating composition.

2. The vaccine or immune stimulating composition of claim 1 wherein the adjuvant comprises a mineral oil.

3. The vaccine or immune stimulating composition of claim 1 or 2, wherein the adjuvant comprises a mineral oil as identified by CAS no: 8042-47-5 (EC no: 232-455-8), EC no: 932-078-5, EC no: 934-954-2, or EC no: 934-965-3.

4. The vaccine or immune stimulating composition of claim 3, wherein the adjuvant comprises the mineral oil identified by CAS no: 8042-47-5.

5. The vaccine or immune stimulating composition of claim 3, wherein the adjuvant, comprises the mineral oil identified by EC no: 932-078-5.

6. The vaccine or immune stimulating composition of any one of claims 1 to 4, wherein the adjuvant consists of the mineral oil identified by CAS no: 8042-47-5.

7. The vaccine or immune stimulating composition of any one of the preceding claims, wherein the immunogen is selected from the group consisting of: a plurality of killed Mycobacterium avium subspecies paratuberculosis organisms, a plurality of live attenuated Mycobacterium avium subspecies paratuberculosis organisms, a cell lysate formed from a plurality of Mycobacterium avium subspecies paratuberculosis organisms and one or more peptides or polypeptides having the sequence of an Mycobacterium avium subspecies paratuberculosis protein.

8. The vaccine or immune stimulating composition of any one of the preceding claims, wherein the immunogen is a plurality of killed Mycobacterium avium subspecies paratuberculosis organisms.

9. The vaccine or immune stimulating composition of claim 8, wherein the plurality of killed organisms includes a mixture of organisms from different strains of Mycobacterium avium subspecies paratuberculosis.

10. The vaccine or immune stimulating composition of claim 9, wherein the different strains are selected from strains 316F, K10, Telford and CM 00/416.

11. The vaccine or immune stimulating composition of claim 8, wherein the plurality of killed organisms are exclusively from the strain Mycobacterium avium subspecies paratuberculosis Telford.

12. The vaccine or immune stimulating composition of claim 8, wherein the plurality of killed organisms are exclusively from the strain Mycobacterium avium subspecies paratuberculosis K10 or 316F.

13. The vaccine or immune stimulating composition of claim 8, wherein the plurality of killed organisms are exclusively from the strain Mycobacterium avium subspecies paratuberculosis CM 00/416.

14. The vaccine or immune stimulating composition of any one of the preceding claims wherein the composition is an emulsion comprising the immunogen and adjuvant.

15. The vaccine or immune stimulating composition of any one of the preceding claims, wherein the composition further comprises an emulsifier for emulsifying the immunogen and the adjuvant.

16. The vaccine or immune stimulating composition of claim 15, wherein the emulsifier is blended with the adjuvant in an oil phase prior to combination with an aqueous phase comprising the immunogen.

17. The vaccine or immune stimulating composition of claim 15 or 16, wherein the emulsifier is of animal or vegetable origin.

18. The vaccine or immune stimulating composition of any one of claims 15 to 17, wherein the emulsifier is selected from the group consisting of: a mannide monooleate, a polyoxyethylene ether; a polyoxyethylene sorbitan-fatty acid ester, a polyoxyethylene ether; a fatty acid diethanolamide; a fatty acid monoethanolamide; a fatty acid monisopropanolamide; an alkyl amine oxide; an N-acyl amine oxide; and a N-alkoxyalkyl amine oxide.

19. The vaccine or immune stimulating composition of claim 18, wherein the emulsifier is a mannide monooleate

20. The vaccine or immune stimulating composition of claim 19, wherein the mannide monooleate is of vegetable origin and is identified by CAS no: 25339-93-9 or by CAS no: 9049-98-3.

21. A vaccine or immune stimulating composition comprising, consisting or consisting essentially of:
an immunogen for providing an immune response to Mycobacterium avium subspecies paratuberculosis in a subject; the immunogen being provided in an aqueous medium and wherein the immunogen is in the form of a plurality of killed Mycobacterium avium subspecies paratuberculosis organisms,
an adjuvant for potentiating the immune response to the immunogen, wherein the adjuvant comprises or consists of a mineral oil identified by CAS no: 8042-47-5; and
an emulsifier for emulsifying the immunogen and adjuvant;
thereby forming a vaccine or immune stimulating composition.

22. The vaccine or immune stimulating composition of claim 21 wherein the emulsifier is a mannide monooleate, preferably of vegetable origin.

23. A method of reducing the severity of infection with Mycobacterium avium subspecies paratuberculosis in a subject in need thereof, the method comprising administering to the subject, a vaccine or immune stimulating composition of any one of claims 1 to 22.

24. A method for inducing an immune response in a subject to Mycobacterium avium subspecies paratuberculosis, the method comprising administering to the subject a vaccine or immune stimulating composition of any one of claims 1 to 22.

25. A method of reducing the severity of one or more signs of infection with Mycobacterium avium subspecies paratuberculosis or Johne's Disease in a subject, the method comprising administering to the subject a vaccine or immune stimulating composition of any one of claims 1 to 22.

26. The method of claim 25 wherein the one or more signs of infection or disease is selected from the group consisting of: faecal shedding of Mycobacterium avium subspecies paratuberculosis, tissue load of Mycobacterium avium subspecies paratuberculosis, gross pathology consistent with Mycobacterium avium subspecies paratuberculosis infection, histopathology consistent with Mycobacterium avium subspecies paratuberculosis infection, diarrhoea, weight loss, hypoalbuminemia, bottle jaw, and cachexia.

27. The method of any one of claims 23 to 26 wherein the subject is a ruminant selected from the group consisting of cows, sheep, goats, camelid and deer.

28. A method of reducing the likelihood of transmission of Mycobacterium avium subspecies paratuberculosis in a population of ruminants, the method comprising administering to one or more individuals in the population, a vaccine or immune stimulating composition according to any one of claims 1 to 22.

29. Use of the vaccine or immune stimulating composition of any one of claims 1 to 22, for reducing the severity of an infection with Mycobacterium avium subspecies paratuberculosis in a subject.

30. Use of the vaccine or immune stimulating composition of any one of claims 1 to 22, for stimulating an immune response in a subject to Mycobacterium avium subspecies paratuberculosis.

31. Use of the vaccine or immune stimulating composition of any one of claims 1 to 22, for reducing the severity of one or more signs of infection with Mycobacterium avium subspecies paratuberculosis or Johne's Disease in a subject.

32. The use of claim 31, wherein the one or more signs of infection or disease is selected from the group consisting of: faecal shedding of Mycobacterium avium subspecies paratuberculosis, tissue load of Mycobacterium avium subspecies paratuberculosis, gross pathology consistent with Mycobacterium avium subspecies paratuberculosis infection, histopathology consistent with Mycobacterium avium subspecies paratuberculosis infection, diarrhoea, weight loss, hypoalbuminemia, bottle jaw, and cachexia.

33. The use of any one of claims 29 to 32 wherein the subject is a ruminant selected from the group consisting of cows, sheep, goats, camelid and deer.

34. Use of the vaccine or immune stimulating composition of any one of claims 1 to 22 for reducing the likelihood of transmission of Mycobacterium avium subspecies paratuberculosis in a population of ruminants.

35. The vaccine or immune stimulating composition of any one of claims 1 to 22 for reducing the severity of an infection with Mycobacterium avium subspecies paratuberculosis in a subject.

36. The vaccine or immune stimulating composition of any one of claims 1 to 22 for stimulating an immune response in a subject to Mycobacterium avium subspecies paratuberculosis.

37. The vaccine or immune stimulating composition of any one of claims 1 to 22, for reducing the severity of one or more signs of infection with Mycobacterium avium subspecies paratuberculosis or Johne's Disease in a subject.

38. The vaccine or immune stimulating composition for the use of claim 37, wherein the one or more signs of infection or disease is selected from the group consisting of: faecal shedding of Mycobacterium avium subspecies paratuberculosis, tissue load of Mycobacterium avium subspecies paratuberculosis, gross pathology consistent with Mycobacterium avium subspecies paratuberculosis infection, histopathology consistent with Mycobacterium avium subspecies paratuberculosis infection, diarrhoea, weight loss, hypoalbuminemia, bottle jaw, and cachexia.

39. The vaccine or immune stimulating composition for the use of any one of claims 35 to 38, wherein the subject is a ruminant selected from the group consisting of cows, sheep, goats, camelid and deer.

40. The vaccine or immune stimulating composition of any one of claims 1 to 22 for reducing the transmission of Mycobacterium avium subspecies paratuberculosis in a population of ruminants.

41. A kit for use in a method of:
reducing the severity of an infection with Mycobacterium avium subspecies paratuberculosis in a subject;
reducing the severity of one or more signs of Johne's Disease in a subject;
inducing an immune response to Mycobacterium avium subspecies paratuberculosis in a subject; and/or
reducing the transmission of Mycobacterium avium subspecies paratuberculosis within a population of ruminants;
wherein the kit comprises:
an immunogen for providing an immune response to Mycobacterium avium subspecies paratuberculosis in the individual,
an adjuvant for potentiating the immune response to the immunogen, in the individual, wherein the adjuvant comprises, consists or consists essentially of a mineral oil as identified by CAS no: 8042-47-5;
optionally wherein the kit comprises instructions for the use of the components.
